(19) United States
(12) Patent Application Publication
(10) Pub. No.: US 2025/0029720 A1
(43) Pub. Date: Jan. 23, 2025

(54) SYSTEMS AND METHODS FOR MAKING MEDICAL DECISIONS BASED ON MULTIMODAL DATA

(71) Applicant: Shanghai United Imaging Intelligence Co., Ltd., Shanghai (CN)

(72) Inventors: Shanhui Sun, Lexington, MA (US); Zhang Chen, Brookline, MA (US); Xiao Chen, Lexington, MA (US); Yikang Liu, Cambridge, MA (US); Lin Zhao, Billerica, MA (US); Terrence Chen, Lexington, MA (US); Arun Innanje, Lexington, MA (US); Abhishek Sharma, Boston, MA (US); Wenzhe Cui, Cambridge, MA (US); Xiao Fan, Cambridge, MA (US)

(73) Assignee: Shanghai United Imaging Intelligence Co., Ltd., Shanghai (CN)

(21) Appl. No.: 18/225,009

(22) Filed: Jul. 21, 2023

(51) Int. Cl. G16H 50/20; G16H 50/70

(52) U.S. Cl. CPC G16H 50/20; G16H 50/70

(57) ABSTRACT

Disclosed herein are deep-learning based systems, methods, and instrumentalities for medical decision-making. A system as described herein may implement an artificial neural network (ANN) that may include multiple encoder neural networks and a decoder neural network. The multiple encoder neural networks may be configured to receive multiple types of patient data (e.g., text and image based patient data) and generate respective encoded representations of the patient data. The decoder neural network (e.g., a transformer decoder) may be configured to receive the encoded representations and generate a medical decision, a medical summary, or a medical questionnaire based on the encoded representations. In examples, the decoder neural network may be configured to implement a large language model (LLM) that may be pre-trained for performing the aforementioned tasks.

### BACKGROUND

In clinical practices, physicians make diagnostic and/or treatment decisions for a patient based on multimodal information including, for example, lab results, medical scan images, descriptions of symptoms experienced by the patient, medical histories of the patient, and/or demographic information of the patient (e.g., age, gender, race, etc.). With the advancement of machine-learning technologies and the ever increasing strain on medical resources, it may be desirable to train an artificial neural network using large amounts of multimodal patient data such that the artificial neural network may acquire human-like reasoning and decision-making capabilities in the medical field.

### SUMMARY

Disclosed herein are systems, methods, and instrumentalities associated with making medical decisions, providing medical summaries, and/or answering medical inquiries based on multimodal patient data. According to embodiments of the present disclosure, a system may include one or more computers and one or more storage devices storing instructions that, when executed by the one or more computers, may cause the one or more computers to implement an artificial neural network (ANN). The ANN may include multiple encoder neural networks and a decoder neural network. The multiple encoder neural networks may be configured to receive respective types of patient data (e.g., multimodal patient data) and generate respective encoded representations (e.g., embeddings) of the multimodal patient data, wherein the multimodal patient data may include at least a first type comprising a description of symptoms experienced by a patient, and a second type comprising one or more of a test result of the patient, a medical history of the patient, or demographic information about the patient. The decoder neural network may be configured to receive the encoded representations of the multimodal patient data and generate an output based on the encoded representations, wherein the output may include at least one of a medical decision (e.g., a medical scan procedure recommended for the patient), a medical summary (e.g., a textual summary of health conditions), or a medical questionnaire associated with the patient.

In examples, the multiple encoder neural networks may include a first encoder neural network and a second encoder neural network. The first encoder neural network may be configured to receive the first type of patient data and generate at least a first vector representing features extracted from the first type of patient data. Similarly, the second encoder neural network may be configured to receive the second type of patient data and generate at least a second vector representing features extracted from the second type of patient data. In examples, the first encoder neural network and the second encoder neural network may be trained jointly via contrastive learning. During the training, first training data and second training data provided respectively to the first and second encoder neural networks may be treated as a positive pair if the training data belong to a same patient, and as a negative pair if the training data belong to different patients. For the positive pair of training data, the first and second encoder neural networks may be trained to generate respective embeddings that are substantially similar to each other, whereas, for the negative pair of training data, the first and second encoder neural networks may be trained to generate respective embeddings that are substantially different from each other.

In examples, the multi-modal patient data may further include a third type of patient data comprising one or more medical images of the patient, and an encoder neural network may be configured to process the one or more medical images and provide embeddings associated with the one or more medical images to the decoder neural network to generate the medical decision, medical summary, or medical questionnaire described herein. For example, the one or more medical images of the patient may include multiple mammogram images (e.g., digital breast tomosynthesis (DBT) images or full-field digital mammography (FFDM) images) of the patient that may correspond to different views of a breast area of the patient, and the output generated by the decoder neural network may include an indication of whether a medical abnormality (e.g., a lesion) exists in the breast area.

In examples, the decoder neural network (and/or the multiple encoder neural networks) may adopt a transformer architecture (e.g., the decoder and/or encoder neural networks may be transformer neural networks), and the respective encoded representations of the multimodal patient data may be concatenated into a combined representation (e.g., a context vector) before being provided to the decoder neural network. The combined representation may include separators that distinguish the respective encoded representations of the multimodal patient data.

In examples, the decoder neural network may be configured to implement a large language model (LLM) (e.g., with 10 billion or more parameters) that may pre-trained for predicting (e.g., with human-like logics and reasoning) the output based on the respective encoded representations of the multimodal patient data. Using such an LLM, the system described herein may allow a user to post a medical question to the system (e.g., a voice or text-based question), and the system may provide an answer to the question based on a prediction made using the language model.

In examples, the decoder neural network may be configured to determine a distribution of actions (e.g., learned via reinforcement learning) that may be associated with respective reward values, and select, from the distribution, a first action associated with a highest reward value as the medical decision for the patient. In examples, the system may obtain additional data about the patient based on the first action, encode the additional patient data into an additional representation or embedding (e.g., using at least one of the encoder neural networks described herein), and determine, using the decoder neural network, a second action for the patient based at least on the additional representation or embedding.

### BRIEF DESCRIPTION OF THE DRAWINGS

A more detailed understanding of the examples disclosed herein may be had from the following description, given by way of example in conjunction with the accompanying drawing.

FIG. 1 is a simplified block diagram illustrating an example of a neural network system according to one or more embodiments of the present disclosure.

FIG. 2 is a simplified block diagram illustrating an example of contrastive learning according to one or more embodiments of the present disclosure.

FIG. 3 is a simplified block diagram illustrating an example of predicting a medical summary according to one or more embodiments of the present disclosure.

FIG. 4A is a simplified block diagram illustrating an example of sequential decision-making according to one or more embodiments of the present disclosure.

FIG. 4B is a simplified block diagram illustrating another example of sequential decision-making according to one or more embodiments of the present disclosure.

FIG. 5 is a simplified flow diagram illustrating an example process for training an artificial neural network to perform one or more of the tasks described in the present disclosure.

FIG. 6 is a simplified block diagram illustrating an example of an apparatus that may be used to perform at least part of the tasks described in the present disclosure.

### DETAILED DESCRIPTION

The present disclosure is illustrated by way of example, and not by way of limitation, in the figures of the accompanying drawings. A detailed description of illustrative embodiments will now be provided with reference to the figures. Although these embodiments may be described with certain technical details, it should be noted that the details are not intended to limit the scope of the disclosure. Further, while some embodiments may be provided in the context of a clinical decision-making, those skilled in the art will understand that the techniques disclosed in those embodiments may also be applicable to other settings including, for example, a virtual question-and-answer (Q&A) session conducted with the machine-learning model(s) or neural network(s) described herein.

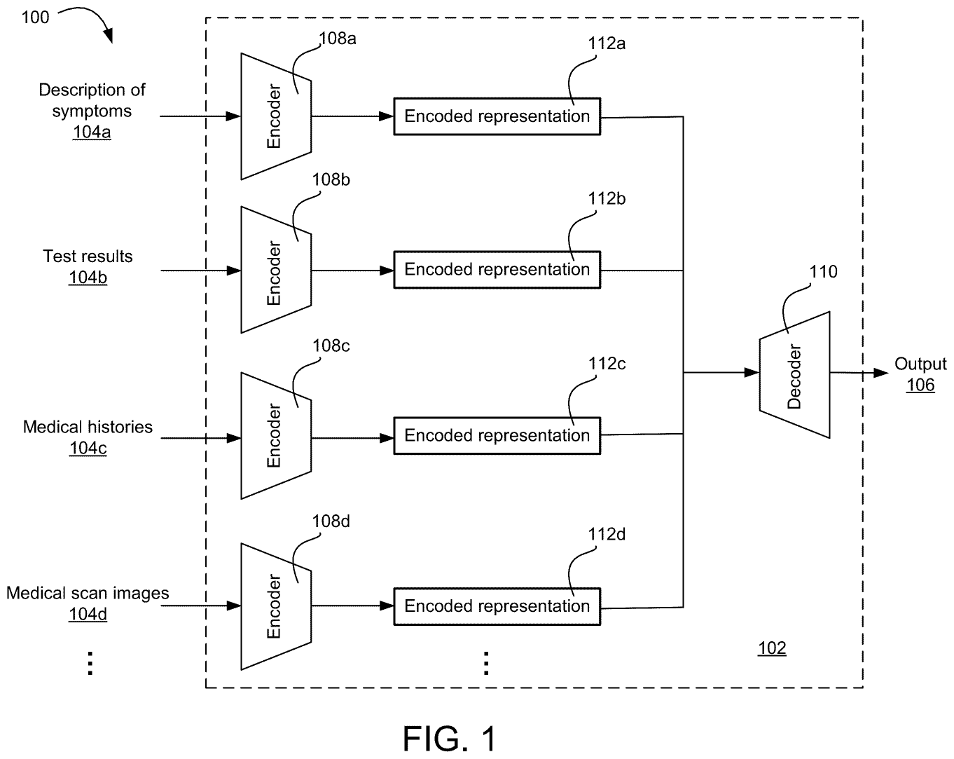

FIG. 1 illustrates an example of a neural network-based system 100 that may be configured to generate an output related to the medical conditions of a patient based on multiple types of patient data and a pre-trained artificial neural network (ANN) 102. The multiple types of patient data may be referred to herein as multimodal patient data and may include textual information about the patient and/or medical images (e.g., including medical videos) of the patient. As shown in FIG. 1, the textual information about the patient may include, for example, a description 104a of symptoms experienced by the patient, test results 104b of the patient (e.g., a lab report such as a blood test report), medical histories 104c of the patient (e.g., the patient's own medical history and/or a family history), demographic information (not shown) about the patient (e.g., age, gender, race, ethnicity, height, weight, etc. of the patient), etc. The medical images 104d of the patient may include, for example, one or more magnetic resonance imaging (MRI) scan images of the patient, one or more computed tomography (CT) scan images of the patient, one or more X-ray scan images of the patient, etc. For instance, the medical images may include full-field digital mammography (FFDM) and/or digital breast tomosynthesis (DBT) images that may correspond to different views of a breast area of the patient, such as, e.g., a right craniocaudal (RCC) view, a left craniocaudal (LCC) view, a right medio-lateral oblique (RMLO) view, and/or a left medio-lateral oblique (LMLO) view of the breast area of the patient. Further, although not shown in FIG. 1, the multimodal patient data may also include audios (e.g., recording of a conversation between the patient and a physician), graphs, and/or other types of data that may indicate the health conditions of the patient.

As shown in FIG. 1, ANN 102 of system 100 may be trained using and/or configured to receive the multimodal patient data, and further configured to generate an output 106 based on the multimodal patient data. In examples, ANN 102 may include multiple encoder neural networks (e.g., encoders 108a-108d) and a decoder neural network 110. Each of the encoder neural networks 108a-108d may be configured to receive a respective type of patient data (e.g., 104a, 104b, 104c or 104d) and generate an encoded representation (e.g., 112a, 112b, 112c, or 112d) of the type of patient data, for example, in the form of one or more vectors (e.g., comprising numeric values). The decoder neural network 110 may be configured to receive the encoded representations of the multimodal patient data (e.g., a concatenation of the encoded representations) and predict the output 106 based on the encoded representations and/or an inquiry (e.g., a question posted by a user of system 100). The predicted output may include, for example, a medical decision about the patient such as a medical procedure (e.g., an MRI or CT scan) to be taken by the patient, whether tumorous cells have been detected in the patient, etc. The predicted output may also include a medical summary (e.g., a textual summary) of the health conditions of the patient generated based on the encoded representations 112a-112d. As an example of a medical decision predicted by the decoder neural network 110, output 106 may include an answer to a question (e.g., about a medical condition) that may be received by the system 100 (e.g., via a user interface) and passed to the decoder neural network 110. For instance, using the decoder neural network 110, system 100 may be capable of engaging a patient in a voice-prompted or text-based question-and-answer (Q&A) session, receiving a description of symptoms experienced by the patient and/or a question posed by the patient, and making a recommendation for an action to be taken by the patient (e.g., obtain a chest X-ray or CT scan). As an example of making a medical decision based on text and image data, the decode neural network 110 may receive respective embeddings (e.g., from the encoder neural networks 108a-108d) of textual data (e.g., description of symptoms, and/or examples, instructions or explanations for the decoder to understand the task at hand) and image data (e.g., multi-view DBT images), and output an indication of whether or which view images may contain a lesion, coordinates and/or size of a bounding box surrounding a lesion, and/or a report containing the aforementioned information.

In examples, the decoder neural network 110 may be configured to generate a medical questionnaire (e.g., as part of the output 106) based on the encoded representations 112a-112d. The questionnaire may be generated in a textual form (e.g., for a patient to fill out) or in an interactive manner with which the system 100 may post questions (e.g., via texts or audios) to the patient, receive answers from the patient (e.g., via texts or audios), and treat the received answers as additional patient data (e.g., by encoding them into a representation) that may be used to derive the next question.

In examples, the multiple encoder neural networks (e.g., 108a-108d shown in FIG. 1) may include one or more text encoder neural networks configured to encode respective types of textual patient data (e.g., patient data 104a, 104b and/or 104c shown in FIG. 1), as well as an image encoder neural network (e.g., a vision transformer) configured to encode image-based patient data (e.g., 104d shown in FIG. 1). In examples, the text encoder neural network and the image encoder neural network may each include a convolutional neural network (CNN) or a recurrent neural network (RNN) comprising one or more convolutional layers, one or more pooling layers, and/or one or more fully-connected layers. Each of the convolutional layers may include multiple kernels or filters with respective weights that may be configured to extract features from an input (e.g., a textual input or an image-based input). The convolution operations may be followed by batch normalization and/or an activation function (e.g., such as a rectified linear unit (ReLu) activation function), and the features extracted by the convolutional layers may be down-sampled via the one or more pooling layers and/or fully-connected layers to obtain a representation of the extracted features, for example, in the form of a feature vector. In the case of an RNN, the network may also be configured to store hidden states associated with the input and feed the hidden states back into the convolutional layers (e.g., via one or more recurrent connections) of the encoder network. This way, the encoder neural network may, during feature encoding, utilize not only the current set of data samples passing through the network, but also previous data samples represented by the hidden states to derive a more accurate representation of the input data.

In examples, one or more (e.g., all) of the encoder neural networks described herein may include an attention (e.g., self-attention) mechanism (e.g., one or more self-attention layers) configured to detect the relationships of different parts of an input data sequence and learn the context (and thus the meaning) of the input data, for example, based on query, key and value vectors or matrices. In these examples, the encoder neural networks may be transformer neural networks.

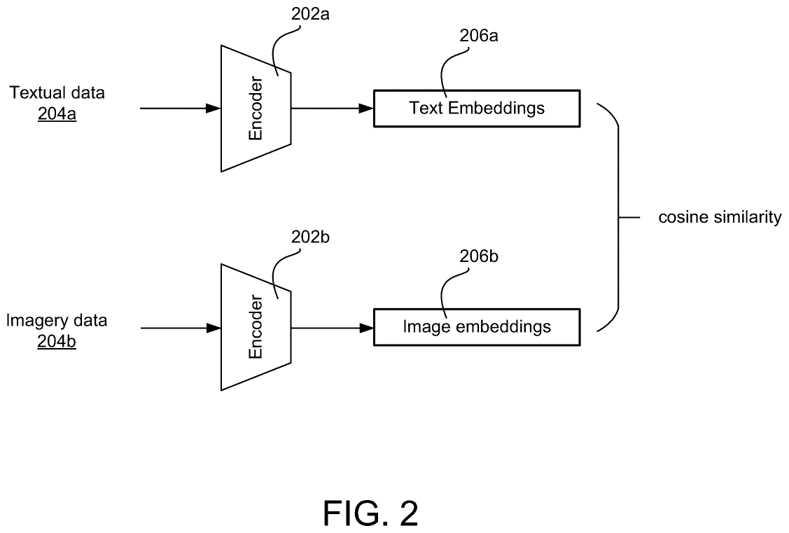

In examples, one or more of the encoder neural networks 108-108d may be trained (e.g., jointly) via contrastive learning. FIG. 2 illustrates such an example, in which a text encoder neural network 202a (e.g., encoder neural network 108a of FIG. 1) and an image encoder neural network 202b (e.g., encoder neural network 108d of FIG. 1) may be trained jointly. As shown in FIG. 2, the training may be conducted based on text-based patient data 204a (e.g., lab results) and image-based patient data 204b (e.g., medical scan images), which may be provided to and processed by the text encoder neural network 202a and image encoder neural network 202b, respectively. During the training, the text-based patient data 204a and image-based patient data 204b belonging to the same patient may be treated as a positive pair, and the respective embeddings (e.g., encoded representations 206a and 206b of FIG. 2) generated by the corresponding encoder neural networks for these data may be forced to be substantially similar, for example, using a contrastive loss function (e.g., a cosine similarity based loss function). This may be because, for example, an accurately predicted text embedding and image embedding should both indicate the presence or non-presence a medical abnormality (e.g., a tumor), given that the text-based patient data 204a and image-based patient data 204b belong to the same patient. Conversely, the text-based patient data 204a and image-based patient data 204b belonging to different patients may be treated as a negative pair during the training, and the respective embeddings generated by the corresponding encoder neural networks for these data may be forced to be substantially different using the contrastive loss function (e.g., since the patients may have different medical conditions).

Referring back to FIG. 1, the encoded representations (e.g., which may also be referred to herein as embeddings) of the multimodal patient data may be concatenated into a combined representation (e.g., a context vector) before the combined representation is passed to the decoder neural network 110 for predicting the output 106. In the combined representation, respective embeddings (e.g., vectors) associated with the multiple modalities may be distinguished from each other using separators (e.g., fixed separators such as “sep” or data type indicating separators such as “dia” for diagnostic test results and “img” for medical images) so that the decoder neural network 110 may determine the respective lengths of the embeddings and decode them accordingly.

In examples, the decoder neural network 110 may adopt a transformer architecture (e.g., with a built-in attention mechanism as described herein and/or a masking mechanism to make the decoder unilateral) and, as such, the decoder neural network may be a transformer decoder. In examples, the decoder neural network 110 may be configured to implement a large language model (e.g., with 10 billion or more parameters) such as an autoregressive language model that may be trained for predicting the output 106 based on the embeddings provided by the encoder neural networks (e.g., based on the concatenated embeddings described above). The large language model (LLM) may be trained on massive amounts of multimodal patient data (e.g., the types of patient data described herein) to learn the statistical relationships of different embeddings and acquire the ability to generate a coherent and contextually relevant output when given a set of patient-specific data (e.g., the multimodal patient data 104a-104d). The number of parameters of the autoregressive language model (e.g., weights of the decoder neural network 110) may be large, in which case the model may become a large language model (LLM). Using such a pre-trained LLM, system 100 shown in FIG. 1 may allow a user of the system to interact with the LLM and obtain medical advice from the LLM. For example, system 100 may be configured to receive a description of medical conditions or symptoms, or a medical question, from the user and generate an answer to the question based on the knowledge or intelligence (e.g., human-like intelligence) acquired by the LLM through training.

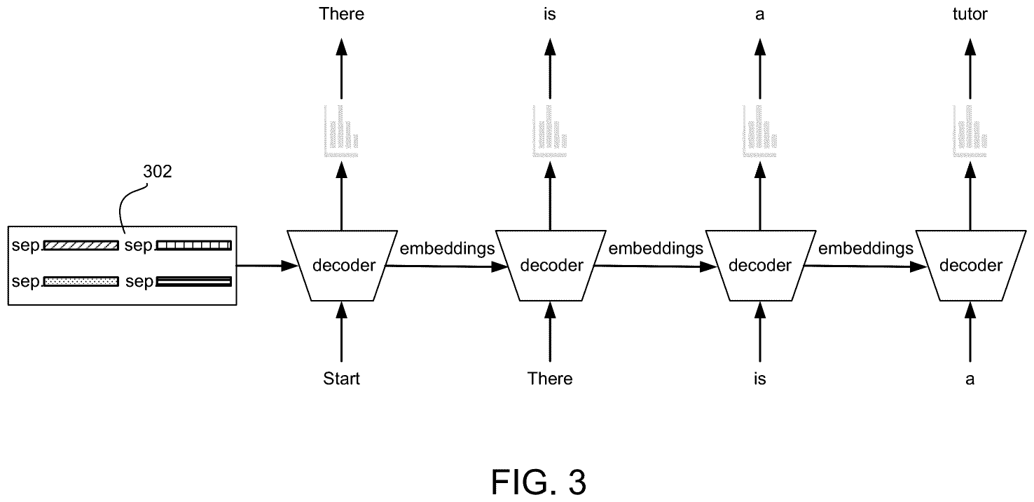

In examples, the output of the decoder neural network 110 may include a medical decision (e.g., whether a specific medical procedure such as an MRI or CT scan should be taken by the patient, whether tumorous cells have been detected in the patient, etc.), in which case the task may be treated as a classification problem (e.g., each class may represent a decision), and the decoder neural network may be trained using a loss function such as a cross-entropy based loss function. In examples, the output of the decoder neural network 110 may include a textual summary or a medical questionnaire generated based on the encoded representations 112a-112d (e.g., a concatenation of the encoded representations), in which case the decoder neural network 110 may be trained via supervised learning. For example, as illustrated by FIG. 3, the decoder neural network 110 (e.g., which may employ a transformer architecture), may be trained to predict a textual summary word by word until the end of a sentence or the end of a summary symbol is predicted. The input to the decoder network may include multimodal embeddings 202 (e.g., a concatenation of encoded embeddings 112a-112d shown in FIG. 1) as well as a word predicted in a previous step. In examples, the input embedding may have a fixed size and, if a certain modality is missing for a patient, the positions corresponding to the embedding for that modality may be filled with zeros. The training may be conducted based on a ground truth summary (e.g., a real diagnostic report) and utilizing a loss function such as a cross-entropy based loss function. The training may also utilize a teacher forcing technique, which may involve feeding a ground truth word to one or more intermediate prediction steps (e.g., rather than applying the ground truth summary as a whole after the final prediction step).

Referring back to FIG. 1 again, the decoder neural network 110 may, in examples, be implemented as a policy gradient neural network for predicting which action a patient may take based on the encoded embeddings 112a-112d. The decoder neural network 110 may learn the action space (e.g., a distribution of potential actions) from which the decoder neural network may select the action for the patient through a training process. In examples, such a training process may be performed using a reinforcement learning technique, with which a policy associated with the reinforcement learning may be defined as following:

$$π ( a \vert s , θ ) {=}^{def} \frac{{e}^{h ( s , a , θ )}}{{\sum }_{b} {e}^{h ( s , b , θ )}}$$

wherein h may represent the policy gradient neural network, s may represent a state input (e.g., the encoded embeddings described herein), and a and b may represent actions that may be taken based on the policy. During the training, a reward (e.g., a numerical reward value) may be determined (e.g., based on reward function) to optimize the neural network for selecting an optimal action at each step. The reward function may be defined, for example, based on the quality of the output generated by the neural network. For instance, if currently collected patient data (e.g., as reflected by the encoded embeddings) is sufficient for producing a satisfactory result (e.g., a good diagnostic evaluation), a positive reward may be given; otherwise, a negative reward may be given. The training of the policy gradient network may be conducted using a suitable policy gradient based training method, such as, e.g., proximal policy optimization. Once trained and given a set of embeddings as described herein (e.g., corresponding to a state), the policy gradient neural network may predict a distribution of potential actions based on the equation shown above (e.g., a softmax), and select the action with the maximum reward value (e.g., maximum softmax value calculated based on the equation). In examples, the decision-making operations of the decoder neural network 110 may be treated as a classification problem (e.g., to predict an action label at each decision step), in which case the training of the neural network may be conducted in a supervised manner based on a cross entropy based loss function (e.g., instead of the reinforcement learning technique described above).

Based on the pre-trained policy gradient neural network described herein, system 100 shown in FIG. 1 may be used for sequential decision-making (e.g., as a Markov decision process) in a medical setting (e.g., a hospital). For example, at a given time spot, system 100 may receive multimodal information about a patient, obtain respective encoded embeddings (e.g., vectors) of the multimodal information (e.g., using one or more of encoders 108a-108d), and predict an action for the patient using the policy gradient neural network (e.g., decoder neural network 110). Based on the predicted action, additional information about the patient may be collected and encoded into an additional embedding (e.g., an additional vector) for predicting another action or medical decision for the patient.

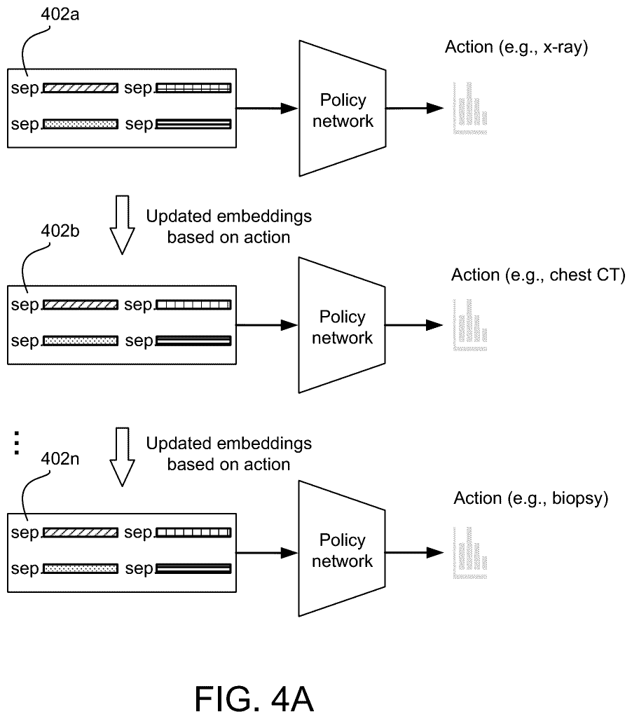

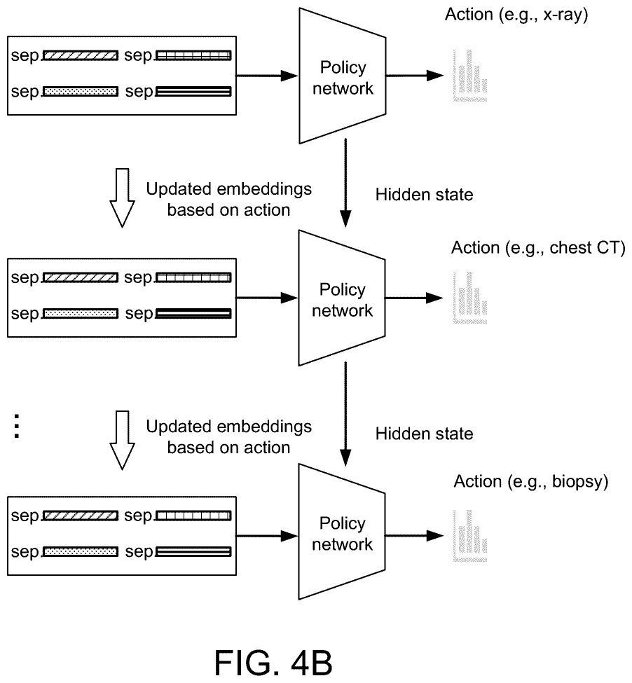

FIG. 4A and FIG. 4B illustrate examples of sequential decision-making using the system (e.g., system 100) described herein. In the example of FIG. 4A, updated embeddings determined based on an action predicted by a policy gradient neural network (e.g., decoder neural network 110 of FIG. 1) may be fed back (e.g., recursively) into the policy gradient neural network for predicting a next action. In the example of FIG. 4B, the policy gradient neural network may include a recurrent neural network (RNN) configured to receive a hidden state associated with a previous decision step as an input (e.g., in addition to updated embeddings) to a current decision step. In either example, the system may predict a first action for a patient (e.g., take an X-ray scan) in accordance with a first set of embeddings (e.g., lab results of the patient, a conversation between the patient and a physician, demographic information about the patient, etc.). Once the patient completes the x-ray scan, the system may obtain the scan image(s) (e.g., from the scanning device or another storage location), encode them into an additional embedding, and provide the additional embedding to the policy gradient neural network. Based on the additional embedding and/or previously encoded embeddings, the system may predict a second action for the patient (e.g., take a chest CT) and repeat the operations described above, for example, until a suitable diagnosis or treatment plan may be determined for the patient.

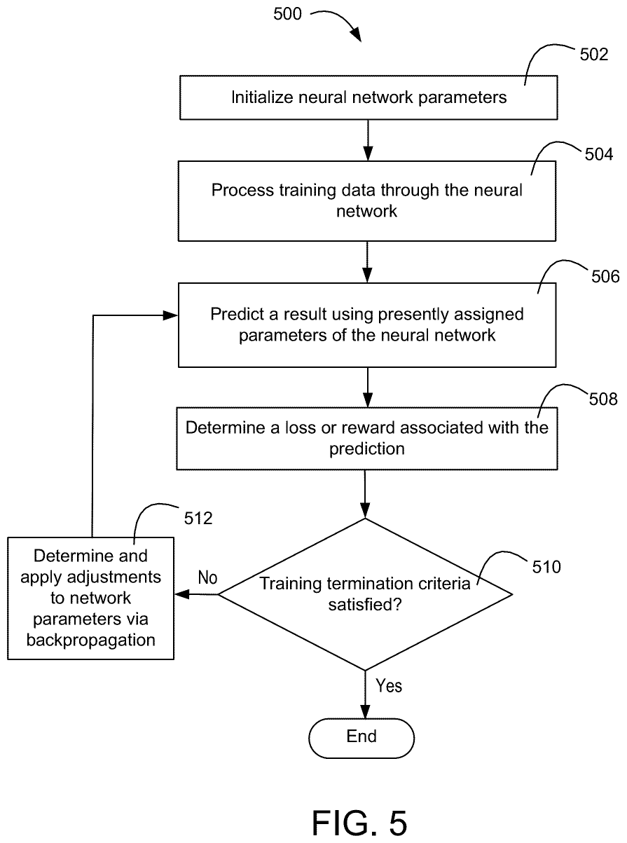

FIG. 5 illustrates an example process 500 for training an artificial neural network (e.g., the encoder neural network or decoder neural network described herein) to perform one or more of the tasks described herein. As shown, training process 500 may include initializing parameters of the neural network (e.g., weights associated with various layers of the neural network) at 502, for example, based on samples from one or more probability distributions or parameter values of another neural network having a similar architecture. The training process may further include processing an input (e.g., patient data if the neural network is an encoder, or an encoded embedding if the neural network is a decider) at 504 using presently assigned parameters of the neural network, and making a prediction for a desired result (e.g., an embedding if the neural network is an encoder, or a medical decision if the neural network is a decoder) at 506. The predicted result obtained at 506 may be evaluated at 508 based on a loss function (e.g., a cross entropy based loss function or a cosine similarity based loss function) or a reward function (e.g., if the training involves reinforcement learning as described herein). The loss or reward associated with the prediction may then be evaluated, at 510, to determine whether one or more training termination criteria are satisfied. For example, in cases where a loss is used as an objective of the training, the training termination criteria may be satisfied if that loss falls below a threshold value or if a change in the loss between two training iterations falls below a threshold value. In cases where a reward is used as an objective of the training, the training termination criteria may be satisfied if the reward reaches a target value (e.g., a maximum reward value defined as part of a policy) or if a change in the reward between two training iterations falls below a threshold value.

If the determination at 510 is that the termination criteria are satisfied, the training may end. Otherwise, the presently assigned network parameters may be adjusted at 512, for example, by backpropagating a gradient descent of the loss or a gradient ascent of the reward through the neural network, before the training returns to 506.

For simplicity of explanation, the training operations are depicted and described herein with a specific order. It should be appreciated, however, that the training operations may occur in various orders, concurrently, and/or with other operations not presented or described herein. Furthermore, it should be noted that not all operations that may be included in the training process are depicted and described herein, and not all illustrated operations are required to be performed.

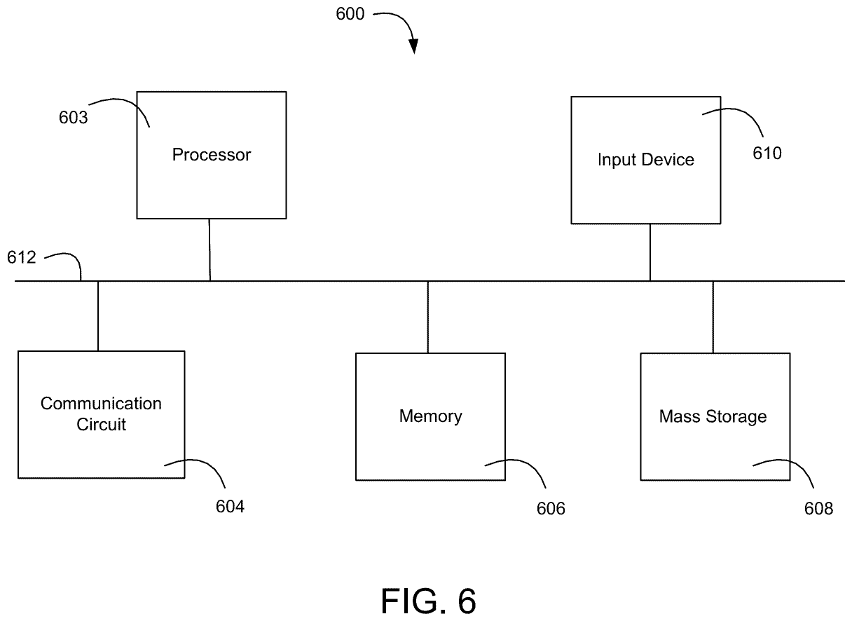

The systems, methods, and/or instrumentalities described herein may be implemented using one or more computers, one or more storage devices, and/or other suitable accessory devices such as display devices, communication devices, input/output devices, etc. FIG. 6 is a block diagram illustrating an example apparatus 600 (e.g., a computer) that may be included in the system described herein and configured to perform at least a part of the functions described herein. As shown in FIG. 6, apparatus 600 may include a processor (e.g., one or more processors) 602, which may be a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. Apparatus 600 may further include a communication circuit 604, a memory 606, a mass storage device 608, an input device 610, and/or a communication link 612 (e.g., a communication bus) over which the one or more components shown in the figure may exchange information.

Communication circuit 604 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). Memory 606 may include a storage medium (e.g., a non-transitory storage medium) configured to store machine-readable instructions that, when executed, cause processor 602 to perform at least a part of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. Mass storage device 608 may include one or more magnetic disks such as one or more internal hard disks, one or more removable disks, one or more magneto-optical disks, one or more CD-ROM or DVD-ROM disks, etc., on which instructions and/or data may be stored to facilitate the operation of processor 602. Input device 610 may include a keyboard, a mouse, a voice-controlled input device, a touch sensitive input device (e.g., a touch screen), and/or the like for receiving user inputs to apparatus 600.

It should be noted that apparatus 600 may operate as a standalone device or may be connected (e.g., networked, or clustered) with other computation devices to perform the functions described herein. And even though only one instance of each component is shown in FIG. 6, a skilled person in the art will understand that apparatus 600 may include multiple instances of one or more of the components shown in the figure.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

What is claimed is:

1. A system comprising one or more computers and one or more storage devices storing instructions that, when executed by the one or more computers, cause the one or more computers to implement an artificial neural network (ANN), wherein the ANN comprises:
multiple encoder neural networks configured to receive respective types of patient data and generate respective encoded representations of the types of patient data, wherein the types of patient data include at least a first type of patient data comprising a description of symptoms experienced by a patient, and a second type of patient data comprising one or more of a test result of the patient, a medical history of the patient, or demographic information about the patient; and
a decoder neural network configured to receive the encoded representations and generate an output based on the encoded representations, wherein the output includes at least one of a medical decision, a medical summary, or a medical questionnaire associated with the patient.

2. The system of claim 1, wherein the types of patient data further include a third type of patient data comprising one or more medical images of the patient.

3. The system of claim 2, wherein the one or more medical images of the patient include multiple mammogram images of the patient that correspond to different views of a breast area of the patient, and wherein the output generated by the decoder neural network includes an indication of whether a medical abnormality exists in the breast area.

4. The system of claim 1, wherein the multiple encoder neural networks include a first encoder neural network and a second encoder neural network, the first encoder neural network configured to encode the first type of patient data into at least a first vector representing features of the first type of patient data, the second encoder neural network configured to encode the second type of patient data into at least a second vector representing features of the second type of patient data.

5. The system of claim 4, wherein the first encoder neural network and the second encoder neural network are trained jointly via contrastive learning and, during the training, first training data and second training data provided respectively to the first encoder neural network and the second encoder neural network are treated as a positive pair if the first training data and second training data belong to a same patient, and as a negative pair if the first training data and second training belong to different patients.

6. The system of claim 1, wherein the decoder neural network includes a transformer neural network.

7. The system of claim 6, wherein the respective encoded representations of the types of patient data are concatenated into a combined representation and provided to the decoder neural network, the combined representation including separators that distinguish the respective encoded representations of the types of patient data.

8. The system of claim 1, wherein the decoder neural network is configured to implement a large language model (LLM) pre-trained for predicting the output based on the respective encoded representations of the types of patient data.

9. The system of claim 8, wherein, when executed by the one or more computers, the instructions stored in the one or more storage devices further cause the one or more computers to receive a medical question and generate an answer to the medical question based on the LLM.

10. The system of claim 1, wherein the decoder neural network is configured to determine a distribution of actions associated with respective reward values and select, from the distribution, a first action associated with a highest reward value as the medical decision for the patient.

11. The system of claim 10, wherein the decoder neural network is trained to learn the distribution of actions via reinforcement learning.

12. The system of claim 10, wherein, when executed by the one or more computers, the instructions stored in the one or more storage devices further cause the one or more computers to obtain additional patient data based on the first action selected by the decoder neural network, encode the additional patient data into an additional representation using at least one of the multiple encoder neural networks, and determine, using the decoder neural network, a second action for the patient based at least on the additional representation.

13. The system of claim 1, wherein the medical decision associated with the patient includes a recommendation of a medical scan procedure for the patient.

14. A method, comprising:
encoding, using respective encoder neural networks, multiple types of patient data into respective encoded representations, wherein the multiple types of patient data include at least at least a first type of patient data comprising a description of symptoms experienced by a patient, and a second type of patient data comprising one or more of a test result of the patient, a medical history of the patient, or demographic information about the patient; and
generating an output based on the encoded representations, wherein the output is generated using a decoder neural network and comprises at least one of a medical decision, a medical summary, or a medical questionnaire associated with the patient.

15. The method of claim 14, wherein the multiple types of patient data further include a third type of patient data comprising one or more medical images of the patient.

16. The method of claim 15, wherein the one or more medical images of the patient include multiple mammogram images of the patient that correspond to different views of a breast area of the patient, and wherein the output generated by the decoder neural network includes an indication of whether a medical abnormality exists in the breast area.

17. The method of claim 14, wherein the decoder neural network includes a transformer neural network.

18. The method of claim 17, wherein the respective encoded representations of the multiple types of patient data are concatenated into a combined representation and provided to the decoder neural network, the combined representation including separators that distinguish the respective encoded representations of the multiple types of patient data.

19. The method of claim 14, wherein the decoder neural network is configured to implement a large language model (LLM) pre-trained for predicting the output based on the respective encoded representations of the multiple types of patient data, and wherein generating the output based on the encoded representations comprises receiving a medical question and generating an answer to the medical question based on the LLM.

20. The method of claim 14, wherein the output generated by the decoder neural network includes a recommendation of a medical scan procedure for the patient.
